(19) United States
(12) Patent Application Publication
(10) Pub. No.: US 2007/0154410 A1
(43) Pub. Date: Jul. 5, 2007

(54) Mouthwash Composition for the Prevention of Dental Diseases

(76) Inventor: Julia Joungmy Kim, Langhorne, PA (US)

Correspondence Address: Julia Joungmy Kim, 711 Daylily Drive, Langhorne, PA, 19047, US

(21) Appl. No.: 11/622,126

(22) Filed: Jan. 11, 2007

(30) Foreign Application Priority Data
Jan. 2, 2006 (KR) 10-2006-0000087

(51) Int. Cl. A61K 8/19

(52) U.S. Cl. 424/49

(57) ABSTRACT

A mouthwash composition for inhibiting the buildup of plaque on dental surfaces and preventing dental diseases consisting essentially of an aqueous solution of alkali metal chlorides as active agents wherein the active agents include 0.5 % to 8.0 % by weight of at least one of the alkali metal chlorides including CaCl₂, MgCl₂ and KCl, with the balance being water.

### FIELD OF THE INVENTION

This invention relates to mouthwash compositions for the prevention of dental diseases, and, in particular, to mouthwash formulations, which upon application to the teeth, inhibit the buildup of plaque on dental surfaces and prevent dental diseases associated with excessive plaque formation.

### BACKGROUND OF THE INVENTION

Dental plaque is present on dental surfaces. It is a by-product of microbial growth, and comprises a dense microbial layer consisting of a mass of microorganisms embedded in a polysaccharide matrix. The danger associated with the formation of plaque on the teeth lies in the tendency of plaque to build up and eventually produce dental diseases such as gingivitis, periodontitis, malodor, dental caries and dental calculus.

It is recognized in dentistry that brushing alone for many individuals will not prevent the formation of significant amounts of plaque. Thus, chemical plaque control, in the form of mouthwash, has been sought as a substitute or supplement to regular brushing.

Many efforts have been made toward finding improved means for retarding plaque accumulation without many of the side effects associated with desquamation and soreness of oral mucosa, objectionable taste, toxicity and imbalance of the oral flora.

For example, mouthwashes containing sodium fluoride were disclosed in U.S. Pat. No. 2,913,373, U.S. Pat. No. 3,975,514, U.S. Pat. No. 4,548,809, and US 20030124068A1. However, the inactivation of fluoride became a problem as the surface active wetting agent was present. Mouthwashes containing various alkali metal compounds were also disclosed: sodium benzoate (WO 9409752); alkali metal hypohalite (US 20020114851A1); chlorine dioxide (CN 1222345); alkali metal phosphate (US 20010002252A1, US 20030007937A1); hydrogen sulfate/carbonate (JP 8113519); cetylpyridium chloride(CPC) (U.S. Pat. No. 6,117,417, U.S. Pat. No. 5,948,390, JP 2004051511). Mouthwashes containing higher alcohol (US 20020064505A1, US 20030175216A1); hydrogen peroxide (CN 1385145); C02 gas bubbles (JP 1275521, JP 2157215) were also disclosed. However, a mouthwash containing alkali metal chlorides including CaCl₂, MgCl₂ and KCl as active agents (not as appendages) for preventing dental diseases has not yet been disclosed.

The mouthwashes described above may retard the accumulation of plaque and calculus to some degree and hinder the development the dental diseases for some periods. However, many people still suffer from dental diseases such as gingivitis, periodontitis, caries or oral malodor even after using such mouthwashes. Some mouthwashes still have side effects such as an objectionable astringent taste, soreness of oral mucosa or imbalance of the oral flora.

It is therefore necessary to develop a mouthwash, which can more effectively inhibit the accumulation of plaque and calculus and thus prevent the above-mentioned dental diseases with fewer side effects.

In view of the foregoing, it is an object of this invention to provide a mouthwash composition which functions to inhibit the buildup of plaque on dental surfaces and to prevent the development of dental diseases associated with excessive plaque formation such as gingivitis, periodontitis and oral malodor.

### BRIEF SUMMARY OF THE INVENTION

In accordance with the foregoing objectives, this invention provides a mouthwash intended for application to dental surfaces for the purpose of inhibiting the buildup of plaque on dental surfaces and to prevent the development of dental diseases associated with excessive plaque formation such as gingivitis (gingival inflammation), periodontitis and oral malodor. This mouthwash is comprised of an aqueous solution of alkali-metal chlorides or alkali-metal ions and chlorine ions as an active agent, with the balance being water.

In preferred embodiments of this invention, the mouthwash composition has an aqueous solution of 0.5% to 8.0% by weight of at least one of the alkali-metal chlorides as an active agent with the balance being water; or has an aqueous solution of 0.25% to 2.0% by weight of at least one of the alkali-metal ions and 0.25% to 6.0% by weight of chlorine ion as active agents with the balance being water.

In further embodiments of this invention, minor effective amounts of colorant, flavorant, sweetener and other additives can be advantageously added in this invention.

In further embodiments of this invention, impurities may be included in this mouthwash.

This invention also provides a method for improving oral hygiene by applying the mouthwash for preventing the further development of plaque on dental surfaces. The mouthwash may be applied to the surface of the teeth by any conventional process. Preferably, however, one should place a comfortable amount of the mouthwash in the oral cavity and then circulate the mouthwash about the mouth with the intention of thoroughly soaking the teeth and gums.

The dramatic reduction in plaque content accomplished through the use of this mouthwash and associated methods of this invention should also serve to prevent plaque-associated dental diseases such as gingivitis, periodontitis and malodor.

Further advantages and objectives of this invention will be apparent from the detailed descriptions of the invention which follows.

### DETAILED DESCRIPTION OF THE INVENTION

The mouthwash formulation of this invention is comprised of an aqueous solution of 0.5% to 8.0% by weight of at least one of the alkali-metal chlorides, including CaCl₂, MgCl₂ and KCl as active agents, with the balance being water.

The alkali-metal chlorides may exist as alkali-metal ions and chlorine ions in a water solution. In view of the foregoing, the mouthwash formulation of this invention also is comprised of an aqueous solution of 0.25% to 2.0% by weight of at least one of the alkali-metal ions, including Ca, Mg, and K, and 0.25% to 6.0% of chlorine ions as active agents, with the balance being water.

If the total content of alkali metal chlorides is below the above-defined range (or if the total contents of alkali-metal ions and chlorine ion are below the minimum), the resulting solution becomes less effective for preventing the accumulation of plaque and plaque-associated dental diseases such as gingivitis (gingival inflammation), periodontitis and oral malodor. If the total content of alkali metal chlorides is beyond the above-defined range (or if the total contents of alkali-metal ions and chlorine ion are above the maximum), the resulting solution has side effects such as objectionable taste or segregation of the active agents.

It has been found that water solutions of some alkali metal chlorides, including CaCl₂, KCl, MgCl₂ or their mixtures being within the above-defined ranges, very effectively reduced dental diseases such as malodor and gingival inflammation, while other alkali metal chlorides such as NaCl had little effect.

The proper amount of alkali-metal chlorides including CaCl₂, KCl, MgCl₂ or their mixtures employed in embodiment of this invention imparts plaque inhibiting properties to the solution. NaCl, NaHCO₃ or Na₂SO₄ does not effectively impart plaque inhibiting properties but may be included in the solution as an appendage. The other chlorides including FeCl₂, ZnCl₂, MnCl₂ or CuCl₂ may also be included as unavoidable impurities.

In addition to the foregoing ingredients, the mouthwash of this invention may include adjuvant ingredients effective to provide desirable flavoring and coloring. The flavorant or colorant employed in this invention is one which should not impede the antibacterial activity of the formulation. The flavorant may include thymol, eucalyptol, menthol, or mint. The mouthwash of this invention may also contain a sweetener such as glycerine, sorbitol, saccharine, dextrose, xylitol or glucose.

The mouthwash is prepared by mixing the alkali-metal chlorides and adjuvant ingredients together in water to form a homogeneous solution of the constituents. The mouthwash is used in a conventional manner: that is, by applying a comfortable amount in the mouth, approximately 10˜30 ml, and rinsing it about the dental surfaces and gums. The mouthwash may be used alone or after a conventional tooth-brushing regimen.

The alkali metal chlorides can be used in dental cream as well as in an aqueous mouthwash solution for inhibiting plaque accumulation and preventing dental diseases.

The manner of making and using the present invention will be illustrated further and not limited by the following detailed examples.

### EXAMPLE 1

The following mouthwashes were formulated.

TABLE 1

| Mouthwash | | | | |
|---|---|---|---|---|
| Formula | Components (wt. %) | | | |
| (1) NaCl | 0.4% | Na | 0.36% Cl | 99.24% H₂O |
| (2) CaCl₂ | 0.4% | Ca | 0.71% Cl | 98.89% H₂O |
| (3) MgCl₂ | 0.4% | Mg | 1.17% Cl | 98.43% H₂O |
| (4) KCl | 0.4% | K | 0.62% Cl | 98.98% H₂O |
| (5) Sea water | 1.07% | Na | 1.93% Cl | 96.5% H₂O |
| | 0.04% | K | 0.27% SO₄ | |
| | 0.04% | Ca | | |
| | 0.11% | Mg | | |

Each alkali-metal chloride was weighed on a microbalance and then slowly added to water with low speed agitation as listed in table 1. Each mouthwash formula had a clear appearance. Formulae (1) and (5) had a salty taste; formulae (2) and (3) had a somewhat bitter taste; and formula (4) was tasteless. For comparison, a conventional mouthwash was also tested.

Six patients, having gingival inflammation and malodor, were administered the mouthwashes of table 1 and were instructed to use the gargles in following manner:
a. Brush teeth.
b. Use about 20 ml of a mouthwash after brushing. Rinse around the mouth for about 3 minutes with a mouthwash and then rinse the mouth with water.
c. Repeat steps (a) and (b) twice daily.

All six patients were examined prior to and after usage of the mouthwash for two weeks. The results are summarized in table 2.

TABLE 2

| | Reduction of Ginival | Reduction of Oral |
|---|---|---|
| | Inflammation | Malodor |
| (1) NaCl | Δ | Δ |
| (2) CaCl₂ | ⊙ | ⊙ |
| (3) MgCl₂ | ⊙ | ⊙ |
| (4) KCl | ⊙ | ⊙ |
| (5) Sea water | Δ | ◯ |
| (0) Conventional gargle | Δ | ◯ |

⊙: very effective,
◯: effective,
Δ: minimally effective,
X: not effective

The foregoing exhibits that the mouthwash formulae (2), (3) and (4) were effective in significantly reducing both gingival inflammation and oral malodor over the test period, while formulae (1), (5) and (0) were not as effective in reducing both gingival inflammation and oral malodor.

### EXAMPLE 2

Since the formulae (2), (3) and (4) were effective, solutions of various ranges of CaCl₂, MgCl₂ and KCl were formulated to test the effectiveness of reducing both gingival inflammation and oral malodor:

TABLE 3

| Mouthwash | Components (wt. %) | | | | |
|---|---|---|---|---|---|
| Formula | Ca | Mg | K | Cl | H₂O |
| (21) CaCl₂ | 0.01 | | | 0.02 | 99.97 |
| (22) CaCl₂ | 0.1 | | | 0.18 | 99.72 |
| (23) CaCl₂ | 0.25 | | | 0.45 | 99.30 |
| (24) CaCl₂ | 0.5 | | | 0.89 | 98.61 |
| (25) CaCl₂ | 1.0 | | | 1.78 | 97.22 |
| (26) CaCl₂ | 2.0 | | | 3.55 | 94.45 |
| (27) CaCl₂ | 5.0 | | | 8.88 | 86.12 |
| (31) MgCl₂ | | 0.01 | | 0.03 | 99.96 |
| (32) MgCl₂ | | 0.1 | | 0.29 | 99.4 |
| (33) MgCl₂ | | 0.25 | | 0.73 | 99.02 |
| (34) MgCl₂ | | 0.5 | | 1.46 | 98.04 |
| (35) MgCl₂ | | 1.0 | | 2.92 | 96.08 |
| (36) MgCl₂ | | 2.0 | | 5.84 | 92.16 |
| (37) MgCl₂ | | 5.0 | | 14.59 | 80.41 |
| (41) KCl | | | 0.01 | 0.01 | 99.98 |
| (42) KCl | | | 0.1 | 0.09 | 99.81 |
| (43) KCl | | | 0.25 | 0.23 | 99.52 |
| (44) KCl | | | 0.5 | 0.45 | 99.05 |
| (45) KCl | | | 1.0 | 0.91 | 98.09 |
| (46) KCl | | | 2.0 | 1.81 | 97.19 |
| (47) KCl | | | 5.0 | 4.53 | 90.47 |

The mouthwashes in table 3 were applied to patients having gingival inflammation and oral malodor in the same manner as described in example 1. The test results are summarized in table 4.

TABLE 4

| | Reduction | Reduction | |
|---|---|---|---|
| Mouthwash | of gingival | of oral | |
| Formula | inflammation | malodor | Remarks |
| (21) CaCl₂ | X | X | |
| (22) CaCl₂ | ◯ | ◯ | |
| (23) CaCl₂ | ⊙ | ⊙ | |
| (24) CaCl₂ | ⊙ | ⊙ | |
| (25) CaCl₂ | ⊙ | ⊙ | |
| (26) CaCl₂ | ⊙ | ⊙ | |
| (27) CaCl₂ | ◯ | ◯ | Too bitter |
| (31) MgCl₂ | X | X | |
| (32) MgCl₂ | Δ | Δ | |
| (33) MgCl₂ | ⊙ | ⊙ | |
| (34) MgCl₂ | ⊙ | ⊙ | |
| (35) MgCl₂ | ⊙ | ⊙ | |
| (36) MgCl₂ | ⊙ | ⊙ | |
| (37) MgCl₂ | ◯ | ◯ | Too bitter |
| (41) KCl | X | X | |
| (42) KCl | Δ | Δ | |
| (43) KCl | ⊙ | ⊙ | |
| (44) KCl | ⊙ | ⊙ | |
| (45) KCl | ⊙ | ⊙ | |
| (46) KCl | ⊙ | ⊙ | |
| (47) KCl | ◯ | ◯ | segregation |

⊙: very effective,
◯: effective,
Δ: minimally effective,
X: not effective

As shown in table 4, there was no or little effect in reducing gingival inflammation and oral malodor when the alkali-metal ion (Ca, Mg or K) content was less than 0.1 weight percent in aqueous solution. When the alkali-metal ion contents were in the ranges of 0.25 wt. % to 2.0 wt. % with the appropriate contents (0.25 wt. % to 6.0 wt. %) of chlorine in aqueous solution, the mouthwash solutions were very effective in reducing gingival inflammation and oral malodor. However, when the alkali-metal ion contents were beyond 2.0 wt. % with the appropriate contents of chlorine in aqueous solution, the gargle solutions were less effective and very bitter in taste.

### EXAMPLE 3

Mixtures of alkali metal chlorides were also formulated as follows:

TABLE 5

| | |
|---|---|
| Mouthwash | Components (wt. %) |

| Formula | Ca | Mg | K | Na | Cl | H₂O |
|---|---|---|---|---|---|---|
| (61) | 0.01 | 0.01 | 0.01 | | 0.06 | 99.91 |
| (62) | 0.01 | 0.01 | 0.01 | 1.0 | 1.60 | 97.34 |
| (63) | 0.1 | 0.1 | 0.1 | | 0.56 | 99.14 |
| (64) | 0.1 | 0.1 | 0.1 | 1.0 | 2.10 | 96.60 |
| (65) | 0.3 | 0.3 | 0.4 | | 1.77 | 97.23 |
| (66) | 0.3 | 0.3 | 0.4 | 1.0 | 3.31 | 94.69 |
| (67) | 1.0 | 1.0 | 1.0 | | 5.61 | 91.39 |

The mouthwashes in table 5 were applied to patients having gingival inflammation and oral malodor with the same manner as described in example 1. The test results are summarized in table 6.

TABLE 6

| | Reduction | Reduction | |
|---|---|---|---|
| Mouthwash | of gingival | of oral | |
| Formula | inflammation | malodor | Remarks |
| (61) | Δ | Δ | |
| (62) | Δ | Δ | |
| (63) | ⊙ | ⊙ | |
| (64) | ⊙ | ⊙ | |
| (65) | ⊙ | ⊙ | |
| (66) | ⊙ | ⊙ | |
| (67) | ◯ | ◯ | Too bitter |

⊙: very effective,
◯: effective,
Δ: minimally effective,
X: not effective

As shown in table 6, there was little effect in reducing gingival inflammation and oral malodor when the total contents of mixtures of Ca, Mg and K ions excluding Na ions were less than 0.1 weight percent in aqueous solution. When the total contents of mixtures of Ca, Mg and K ions were in the ranges of 0.25 wt. % to 2.0 wt. % with the appropriate contents of chlorine in aqueous solution, the gargle solutions were effective in reducing gingival inflammation and oral malodor. However, the total contents of mixtures of alkali-metal ions did not count Na ions, because Na ions were not effective in reducing gingival inflammation and oral malodor. When the alkali-metal ion contents excluding Na were beyond 2.0 wt. % with the appropriate contents of chlorine in aqueous solution, the mouthwash solutions were less effective and very bitter in taste.

### EXAMPLE 4

To improve the color and taste of the mouthwash, some colorant, flavor compound and sweetener were added as follows:

TABLE 7

| | Components (wt. %) | | | |
|---|---|---|---|---|
| Mouthwash | Base | Colorant, FDC | Flavorant, | Sweetener, |
| formula | solution | Green3 | Menthol | Saccharin |
| (2) CaCl₂ | (2) | — | — | — |
| (4) KCl | (4) | — | — | — |
| (71) | (2) | — | — | 0.05 |
| (72) | (2) | 0.2 | — | 0.05 |
| (73) | (4) | — | 0.04 | — |
| (74) | (4) | 0.2 | 0.04 | — |
| (75) | (4) | 0.2 | 0.04 | 0.03 |

The mouthwashes in table 7 were applied to patients having gingival inflammation and oral malodor with the same manner as described in example 1. The test results are summarized in table 8.

TABLE 8

| | Reduction | Reduction | |
|---|---|---|---|
| Mouthwash | of gingival | of oral | |
| Formula | inflammation | malodor | Taste Reaction |
| (2) | ⊙ | ⊙ | Fair |
| (4) | ⊙ | ⊙ | Good |
| (71) | ⊙ | ⊙ | Good |
| (72) | ⊙ | ⊙ | Good |
| (73) | ⊙ | ⊙ | Good |
| (74) | ⊙ | ⊙ | Good |
| (75) | ⊙ | ⊙ | Good |

⊙: very effective,
◯: effective,
Δ: minimally effective,
X: not effective

As shown in table 8, the addition of minor amounts of colorant, flavorant, and/or sweetener to the aqueous solution including alkali-metal chlorides did not affect the effectiveness of the mouthwash, but improved the taste.

### EXAMPLE 5

To determine the effect of the mouthwash on the plaque and calculus accumulation, the following mouthwash formula was prepared. For comparison, a conventional mouthwash and drinking water were also prepared.

TABLE 9

| Mouthwash Formula | Components (wt. %) |
|---|---|
| (2) CaCl₂ | 0.4% Ca, 0.71% Cl, 98.89% H₂O |
| (6) Conventional | 0.04% NaF, 11% Alcohol. sorbitol, flavors, water |
| (0) Water | 100% H₂O |

The mouthwashes in table 9 were applied to patients having gingival inflammation and oral malodor for six months with the same manner as described in example 1. The test results are summarized in table 10.

TABLE 10

| | Reduction | Reduction | |
|---|---|---|---|
| Mouthwash | of gingival | of oral | Accumulation |
| Formula | inflammation | malodor | of Calculus |
| (2) | Very effective | Very effective | None |
| (6) | Minimally effective | Effective | Medium |
| (0) | Minimally effective | Minimally effective | Heavy |

As shown in table 10, the patient who did not use any mouthwash over the test period experienced severe gingival inflammation, oral malodor and heavy calculus accumulation, while the patient who used conventional mouthwash had somewhat reduced calculus accumulation and malodor but still suffered from gingival inflammation. On the other hand, the patient who used the aqueous solution of CaCl₂ (formula 2) for six months had very healthy teeth without gingival inflammation, oral malodor, or calculus accumulation.

It has been found, as described in the foregoing, that a mouthwash composition consisting essentially of an aqueous solution of 0.25% to 2.0% by weight of at least one of the alkali-metal ions including Ca, Mg and K, and from 0.25% to 6.0% by weight of chlorine ions as active agents (or from 0.5% to 8.0% by weight of at least one of alkali metal chlorides including CaCl₂, MgCl₂ and KCl as an active agent) with the balance being water, effectively inhibited the buildup of plaque on dental surfaces and prevented the dental diseases.

This invention has been described in terms of specific embodiments set forth in detail herein, but it should be understood that these are by way of illustration and the invention is not necessarily limited thereto. Modifications and variations will be apparent from disclosure and may be restored to without departing from the spirit of the invention as those of skill in the art will readily understand. Accordingly, such variations and modifications are considered to be within the purview and scope of the invention and the following claims.

### LIST OF REFERENCES

### References Cited

### U.S. Patent Documents

2,913,373 11/1959W. S. Weisz
3,975,514 8/1976 G. F. Weisz
4,548,809 10/1985P. S. T. Fung
20030124068A1 7/2003 Y. Atsushi
20020114851A1 8/2002 J. Camper
20010002252A1 5/2001 A. Gallopo
20030007937A1 1/2003 T. Lawlor
6,117,417 9/2000 M. A. Wicks
5,948,390 9/1999 D. Nelson
20020064505A1 5/2002 M. Rosenberg
20030175216A1 9/2003 M. Rosenberg

### FOREIGN PATENT DOCUMENTS

9,409,752 7/1995 World
1,222,345 7/1999 China
8,113,519 5/1996 Japan
2004051511A 2/2004 Japan
1,385,145 12/2002China
1,275,521 11/1989Japan
2,157,215 6/1990 Japan

### List of tables:

Table 1. Various mouthwash compositions
Table 2. Effects of mouthwashes listed in table 1 on periodontal diseases
Table 3. Various ranges of alkali metal chlorides in aqueous solution
Table 4. Effects of mouthwashes listed in table 3 on periodontal diseases
Table 5. Various ranges of alkali metal chloride mixtures in aqueous solution
Table 6. Effects of mouthwashes listed in table 5 on periodontal diseases
Table 7. Mouthwash compositions with various additives
Table 8. Effects of mouthwashes listed in table 7 on periodontal diseases
Table 9. Various mouthwashes
Table 10. Effects of mouthwashes listed in table 9 used for six months on periodontal diseases

What is claimed is:

1. A mouthwash composition for preventing dental diseases consisting essentially of an aqueous solution of alkali metal chlorides as active agents wherein the active agents include 0.5% to 8.0% by weight of at least one of the alkali metal chlorides including CaCl₂, MgCl₂ and KCl, with the balance being water.

2. The mouthwash according to claim 1 wherein the mouthwash composition further includes 0.01% to 1.0% by weight of at least one of colorant, flavorant, and sweetener.

3. The mouthwash according to claim 1 wherein the mouthwash composition further includes 0.01% to 3.0% by weight of at least one of appendages including NaCl, NaF, or Na₂SO₄, and unavoidable impurities including FeCl₂, ZnCl₂, MnCl₂ or CuC12.

4. The mouthwash according to claim 1 wherein the mouthwash means the aqueous mouthwash solution and the mouthwash composition consisting of alkali metal chlorides as active agents, wherein the active agents include 0.5% to 8.0% by weight of at least one of alkali metal chlorides including CaCl₂, MgCl₂ and KCl, with the balance being water.

5. The mouthwash according to claim 1 wherein the mouthwash means the dentifrice and the mouthwash composition consisting of alkali metal chlorides as active agents, wherein the active agents include 0.5% to 8.0% by weight of at least one of the alkali metal chlorides including CaCl₂, MgCl₂ and KCl, with the balance being dental cream.

6. A mouthwash composition for preventing the dental diseases consisting essentially of an aqueous solution of alkali metal ions and chlorine ions as active agents, wherein the active agents include: 0.25% to 2.0% by weight of at least one of alkali metal ions including Ca, Mg and K, and 0.25% to 6.0% by weight of chlorine ions, with the balance being water.
